(19) United States
(12) Patent Application Publication
(10) Pub. No.: US 2026/0217642 A1
(43) Pub. Date: Jul. 30, 2026

(54) IMPROVED PROCESS FOR DEPROTECTION OF N-FORMYL PROTECTED AMINES

(71) Applicant: BASF SE, Ludwigshafen am Rhein (DE)

(72) Inventors: Martin Ernst, Ludwigshafen am Rhein (DE); Carolin Limburg, Ludwigshafen am Rhein (DE); Kirsten Dahmen, Ludwigshafen am Rhein (DE); Sebastian Haupt, Ludwigshafen am Rhein (DE); Rocco Paciello, Ludwigshafen am Rhein (DE); Ralf Boehling, Ludwigshafen am Rhein (DE); Anika Ritter, Ludwigshafen am Rhein (DE); Thomas Schmidt, Ludwigshafen am Rhein (DE)

(73) Assignee: BASF SE, Ludwigshafen am Rhein (DE)

(21) Appl. No.: 19/141,541

(22) PCT Filed: Dec. 11, 2023
(86) PCT No.: PCT/EP2023/085052

§ 371 Date: Jun. 20, 2025

(30) Foreign Application Priority Data
Dec. 20, 2022 (EP) 22215039.3

(51) Int. Cl. C07C 209/78

(52) U.S. Cl. CPC C07C 209/78

(57) ABSTRACT

The present invention relates to a method for converting an N-formyl-protected amine to the corresponding deprotected amine characterized in that the N-formyl-protected amine is deprotected by decomposition of the formyl group to carbon dioxide and hydrogen in the presence of water and a dehydrogenation catalyst and at a temperature in the range from 10 to 300° C. This method can be employed for efficient deprotection of N-formyl protected amino alcohol intermediates within the process for preparing amino alcohols starting from N-formyl protected amino nitriles by reductive hydrolysis of the nitrile function followed by a deprotection of the N-formyl protected amino alcohol intermediate.

The present invention relates to a method for converting an N-formyl-protected amine to the corresponding deprotected amine characterized in that the N-formyl-protected amine is deprotected by decomposition of the formyl group to carbon dioxide and hydrogen in the presence of water and a dehydrogenation catalyst and at a temperature in the range from 10 to 300° C. This method can be employed for efficient deprotection of N-formyl protected amino alcohol intermediates within the process for preparing amino alcohols starting from N-formyl protected amino nitriles by reductive hydrolysis of the nitrile function followed by a deprotection of the N-formyl protected amino alcohol intermediate.

Amino alcohols according to formula (I),

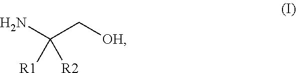

wherein R1 and R2 are independently of each other a hydrogen or an alkyl group having 1 to 4 carbon atoms, such as 2-amino-2-methyl-1-propanol (R1, R2=methyl) are valuable compounds in chemical industry, which are useful organic bases for neutralizing and solubilizing applications, used in toiletries and cosmetics, used in conjunction with fatty acids as a dispersing agent, employed as formaldehyde scavenging and as a wetting agent.

Currently, such amino alcohols are produced technically by the nitration of alkanes, followed by a Henry reaction with formaldehyde and subsequent reduction to the amino alcohol, as shown by the following reaction scheme.

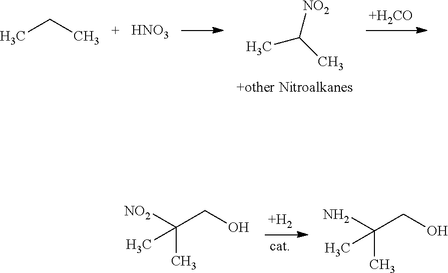

However, the nitration step of this synthesis route is not very selective, and it is associated with safety issues as some nitroalkanes are shock-sensitive and can explode when not treated properly.

To solve these drawbacks, WO 2020/094454 A recently suggested a synthesis route which is based on the reductive nitrile hydrolysis of the corresponding nitrile compound. Central step of this approach is the reductive hydrolysis of the nitrile group of the starting compound. As shown in the following reaction scheme the nitrile is hydrogenated in the presence of a homogeneous transition metal catalyst and water to form the corresponding alcohol and ammonia as a by-product.

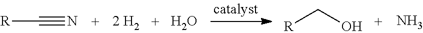

In this approach it is not possible to start the reaction directly with the corresponding aminonitrile because under these reaction conditions such aminonitrile decomposes in a reverse Strecker reaction as shown by the following reaction scheme.

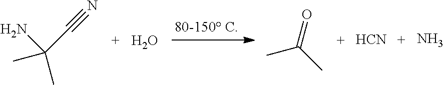

Therefore, according to WO 2020/094454 A, an N-formyl-protected aminonitrile is employed as starting compound and such N-formyl-aminonitrile is converted either (i) directly in a one-step process to the desired amine alcohol or (ii) in a two-step process with the formation of the corresponding N-formyl-amino alcohol as intermediate which is deprotected to the desired amine alcohol in a separate second step, as shown below.

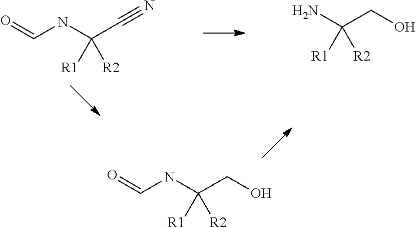

The reductive hydrolysis of the nitrile group for the formation of the corresponding alcohol group within the one-step process or the first step of the two-step process is carried out in the presence of hydrogen and water preferably with a homogeneous transition metal catalyst. Under the appropriate conditions the homogeneous transition metal catalyst catalyses also the cleavage of the formyl group, however, usually with less efficiency. Accordingly, the two-step process allows for a more effective use of the homogeneous transition metal catalyst.

According to WO 2020/094454 A, the deprotection of the amino group in the two-step process can be carried out by means of a hydrolysis or hydrogenation using a hydrolysis or hydrogenation catalyst. The hydrolysis of the N-formyl-protected amine function in the presence of water is usually catalysed by the addition of strong bases or more preferably strong acids such as sulfuric acid or hydrochloric acid and results in the release of formic acid or its salt and the deprotected amine function, possibly in protonated form as ammonium group. The hydrogenation of the N-formyl-protected amine function in the presence of hydrogen is catalysed by hydrogenation catalysts known in the art and results in the release of methanol and the deprotected amine function. Disadvantageously, the addition of strong acids or bases in such a hydrolysis requires their subsequent neutralisation and the separation of the resulting salts. In addition, formic acid is released as an undesirable by-product. The use of strong acids or bases and the release of formic acid also promote corrosion of the device. Cleavage by hydrogenation again requires the use of hydrogen and the associated overpressure device.

It is an object of the invention to provide an advantageous alternative to the deprotection of the N-formyl-protected amino group by hydrolysis or hydrogenation which does not have their above-described disadvantages.

The present invention accordingly relates to the provision of a process for converting an N-formyl-protected amine to the corresponding deprotected amine characterized in that the N-formyl-protected amine is deprotected by decomposition of the formyl group to carbon dioxide and hydrogen in the presence of water and a dehydrogenation catalyst and at a temperature in the range from 10 to 300° C.

In this process the intermediately released formic acid is directly decomposed to carbon dioxide and hydrogen which shifts the equilibrium of the reaction to the formation of the deprotected amine function. Accordingly such process is to be considered as simultaneous release of formic acid and its dehydrogenation to carbon dioxide.

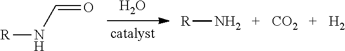

The process of the invention for converting an N-formyl-protected amine to the corresponding deprotected amine is suitable for deprotection of any N-formyl protected aliphatic (linear, branched or cyclic) or aromatic primary or secondary amine, including aliphatic or aromatic amines which exhibit one or more other functional groups as substituents. Accordingly, the N-formyl protected amines are preferably N-formyl protected aliphatic or aromatic primary amines which exhibit one or more substituents selected from the group consisting of hydroxy group, amino group (primary, secondary or tertiary), ether function, thiol group, thioether function, ester function, alkoxy group, nitro group, halogen (chloro, fluoro, bromo or iodo) substituent, nitrile group, keto function and aldehyde group. More preferably, the N-formyl protected amines are N-formyl protected aliphatic or aromatic primary amines which exhibit no substituents or no substituents but one or more hydroxy groups. Particularly preferably, the N-formyl protected amines are N-formyl protected aliphatic primary amines which exhibit one or more hydroxy groups in a 2- (beta), 3- (gamma), or 4- (delta) position related to the carbon atom bearing the N-formyl group (1-position). More particularly preferably, the N-formyl protected amines are N-formyl protected aliphatic primary amines which exhibit one or more hydroxy groups in a 2- (beta) position related to the carbon atom bearing the N-formyl group. Most particularly preferably, the N-formyl protected amine is an N-formyl protected amino alcohol compound of the formula I

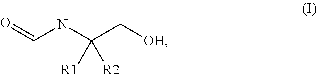

wherein R1 and R2 are independently of each other a hydrogen or an alkyl group having 1 to 4 carbon atoms.

In a particular embodiment, the process of the invention for converting an N-formyl-protected amine to the corresponding deprotected amine is incorporated in a process for manufacturing an amino alcohol compound of the formula II

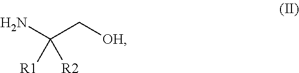

wherein R1 and R2 are independently of each other a hydrogen or an alkyl group having 1 to 4 carbon atoms, characterized in that
(i) in a first step an N-formyl-protected aminonitrile compound of the formula III

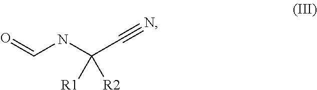

wherein R1 and R2 are independently of each other a hydrogen or an alkyl group having 1 to 4 carbon atoms, is converted to the corresponding N-formyl protected amino alcohol compound of the formula I by the means of a reductive hydrolysis in the presence of water, hydrogen and a homogeneous transition metal catalyst,
(ii) in a subsequent step the resulting N-formyl protected amino alcohol compound of the formula I is deprotected according to the process of the invention for converting an N-formyl-protected amine to the corresponding deprotected amine, optionally after an intermediate purification of the resulting N-formyl protected amino alcohol compound of the formula I from the reaction mixture of the first step.

Accordingly in a particular embodiment, the present invention also relates to a process for manufacturing an amino alcohol compound of the formula II

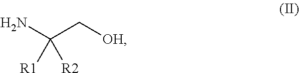

wherein R1 and R2 are independently of each other a hydrogen or an alkyl group having 1 to 4 carbon atoms,
comprising the steps of
a) applying hydrogen to the reaction mixture which comprises an N-formyl-protected aminonitrile compound of the formula III

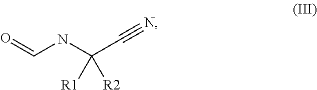

a homogeneous transition metal catalyst and water, to convert the compound of formula III at least partially to the corresponding N-formyl protected amino alcohol compound of the formula I,

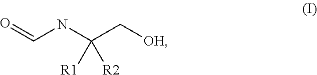

a1) optionally separating the N-formyl protected amino alcohol compound of the formula I resulting from step a) at least partially from the rest of the reaction mixture of step a), and
b) converting the N-formyl-protected amino alcohol compound of the formula I resulting from step a) or the purified N-formyl-protected amino alcohol compound of the formula I resulting from optional step a1) to the corresponding deprotected amino alcohol of formula II,
characterized in that
the N-formyl-protected amino alcohol compound of the formula I is deprotected by decomposition of the formyl group to carbon dioxide and hydrogen in the presence of water and a dehydrogenation catalyst and at a temperature in the range from 10 to 300° C.

In a particular embodiment of the processes for manufacturing an amino alcohol compound of the formula II starting from an N-formyl-protected aminonitrile compound of the formula III according to the invention, the purification of step a1) is mandatory. Preferably, the purification of step a1) comprises the separation of any hydrogen remaining from step a).

Preferably the process for manufacturing an amino alcohol compound of the formula II starting from an N-formyl-protected aminonitrile compound of the formula III according to the invention is carried out with a compound of the formula III in which both residues R1 and R2 are methyl groups. In this case the compound of the formula III is N-(2-cyano-propan-2-yl)formamide and the process results in the formation of as 2-amino-2-methyl-1-propanol as compound of the formula II.

The N-formyl-protected aminonitrile compounds of the formula III are accessible e.g., by the reaction of the corresponding cyanohydrin with formamide in acetic acid as described in WO 2020/094454 A.

Preferably, the deprotection of the N-formyl-protected amine by catalytic decomposition of the formyl group to carbon dioxide and hydrogen within the processes of the invention is carried out without addition of hydrogen.

Preferably, the dehydrogenation catalyst used in the process of the invention is a heterogeneous catalyst which is preferably employed in the form of a fixed bed catalyst. Such heterogeneous dehydrogenation catalysts are well known to the skilled person.

Preferred dehydrogenation catalysts are heterogeneous catalysts with an active material selected from the group consisting of Pt, Pd, Rh, Ru, Ag, Au, Cu, Ni, Co, Fe, Cr, Mo, W, and V, either in metallic form or as a compound, such as oxide or sulfide, including mixtures of such active materials. Particularly preferably, the active material is selected from Pt, Pd, Ag, Cu, Ni, more preferably from Pd, Ag, Cu, Ni. Preferred dehydrogenation catalysts are heterogeneous catalysts having the active material provided on a support material, preferably selected from the group consisting of activated carbon, aluminium oxide, titanium dioxide, zirconium dioxide, silicon dioxide, niobium oxide, vanadium oxide or mixtures thereof, more preferably selected from the group consisting of aluminium oxide, silicon dioxide, zirconium dioxide or mixtures thereof. Preferably, such heterogeneous dehydrogenation catalysts have a BET surface in the range from 1 to 200 m², more preferably in the range from 10 to 150 m², and are employed with a catalyst load in the range from 0.01 to 3 kg/L/h, more preferably in the range from 0.1 to 1 kg/L/h.

The catalytic deprotection of the N-formyl-protected amine according to the invention is carried out in the presence of water at a temperature in the range from 10 to 300° C., preferably in the range from 25 to 270° C., more preferably in the range from 50 to 250° C., particularly preferably in the range from 100 to 230° C., and preferably under a pressure in the range from 0.1 to 300 bara, more preferably in the range from 0.5 to 200 bara, and particularly preferably in the range from 1 to 150 bara. In a particular embodiment, the process of the invention can be carried out at ambient pressure.

The catalytic deprotection of the N-formyl-protected amine according to the invention is carried out in the presence of water, preferably with a weight ratio of the water to the N-formyl-protected amine in the range from 0.1:1 to 100:1, more preferably from 1:1 to 50:1, and particularly preferably from 2:1 to 20:1.

The term “alkyl” as used herein refers to a linear, branched or cyclic hydrocarbon group, without any hetero atom, and preferably saturated, e.g., an ethyl residue. The term “aryl” as used herein refers to a hydrocarbon radical without any hetero atom, but optionally substituted with one or more alkyl radicals, e.g., a phenyl residue or a tolyl residue. The term “arylalkyl” as used herein refers to aryl-comprising residues whose aryl radical is linked via an alkyl chain of at least one carbon atom to the remainder of the molecule, e.g., a benzyl residue. The term “hetero atom” as used herein refers to any atom other than carbon atom or hydrogen atom.

The processes of the invention can be performed continuously, semi-continuously (semi-batch) or discontinuously (batch). Preference is given to continuous processes.

Preferably, in step a) of the process for manufacturing an amino alcohol compound of the formula II starting from an N-formyl-protected aminonitrile compound of the formula III according to the invention, the homogeneous transition metal catalyst is a coordination complex composed of ligands and one or more ruthenium coordination centers. Furthermore, the the homogeneous transition metal catalyst comprises preferably at least one tri-dentate ligand having 3 phosphine atoms. Preferably, this tri-dentate ligand is a ligand of the formula IV or V,

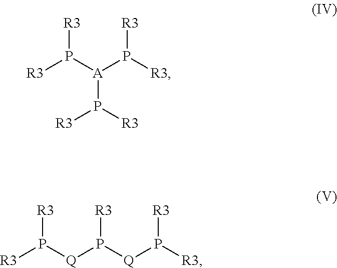

with A being a trivalent alkyl group having 1 to 10 carbon atoms, each Q being independently of each other a divalent alkyl group having 1 to 10 carbon atoms, and each R3 being independently of each other an alkyl group having 1 to 10 carbon atoms, an aryl group having 6 to 12 carbon atoms or an arylalkyl group having 6 to 12 carbon atoms. Preferably, the A residue is a trivalent alkyl group having 1 to 10 carbon atoms. Preferably, the Q residues are divalent alkyl group having 1 to 6 carbon atoms, and preferably both Q residues of the tri-dentate phosphine of formula V are identical. Preferable, the R3 residues are aryl group having 6 to 10 carbon atoms, and preferably all R3 residues of the tri-dentate dentate phosphine of formula IV or V are identical. Particular preferably, such tri-dentate phosphine ligand is selected from the group consisting of 1,1,1-Tris(diphenylphosphinomethyl)ethane (triphos), 1,1,1-Tris(bis(3,5-dimethylphenyl)phosphinomethyl)ethane (triphos-xyl), 1,1,1-Tris(bis(o-tolyl)phosphinomethyl)ethan (triphos-tol), and bis(2-diphenylphosphinoethyl)phenylphosphine (dppepp). Most preferred mandatory ligand of the homogeneous transition metal catalyst is 1,1,1-Tris(diphenylphosphinomethyl)ethane (triphos). In addition to such tri-dentate ligand, the homogeneous transition metal catalyst comprises one or more further ligands, preferably selected from the group consisting of triphenylphosphine (TPP), bis(diphenylphosphino)ethane (dppe), 4,5-Bis(diphenylphosphino)-9,9-dimethylxanthene (xanthphos), Cl⁻, H⁻, CN⁻, acetylacetate, methallyl, 1,5-cyclooctadien, and CO. Particularly preferred further ligands are Cl⁻ and H⁻. Particularly preferred transition metal catalyst comprises no more than one CO ligand, more preferably no CO ligand amongst the further ligands. Particularly preferred homogeneous transition metal catalyst for step a) of the process for manufacturing an amino alcohol compound of the formula II are selected from the group consisting of [Ru₂(Triphos)₂(μ-Cl₃)]Cl, [Ru₂(μ-Cl₃)(triphos)]Cl, [Ru(triphos)(CO)(H)₂], and [Ru(triphos)(methallyl)].

The homogeneous transition metal catalyst of the invention can be prepared by contacting a precursor which contains the transition metal (ruthenium) with the desired ligands. Suitable precursors are ruthenium salts, preferably chloride salts, where the ruthenium cation is complexed with the desired ligands, or different ruthenium complexes (e.g., commercially available complexes) where the original ligands are replaced at least partially by the desired ligands by the means of a ligand replacement reaction.

The preparation of the homogeneous transition metal catalyst of the invention can be carried out by a separate process or in situ within step a), e.g., by adding the Ru containing precursor RuCl₃×3H₂O and the ligand 1,1,1-tris(diphenylphosphinomethyl)ethane to the reaction mixture of step a).

The reductive hydrolysis of step a) is preferably carried out at a temperature in the range from 20 to 200° C., more preferably from 50 to 180° C., and particularly from 100 to 170° C. The hydrogen pressure employed within this step a) is preferably in the range from 0.1 to 400 bar, more preferably from 5 to 200 bar, particularly from 5 to 80 bar. The reductive hydrolysis of step a) is carried out in the presence of water. Preferably the content of water in the reaction mixture of step a) is in the range from 1 to 50% b.w., more preferably in the range from 1 to 30% b.w., and most preferably in the range from 1 to 20% b.w., based on the total reaction mixture. Preferably, the reductive hydrolysis of step a) is carried out in the presence of a solvent, such as an ether, an alcohol or an amide. Preferred is the use of solvents with a comparably high boiling point, e.g., a boiling point which is higher than the amine alcohol compound of the formula I, which is produced by the processes of the invention

The steps of the process of the invention can be carried out as a batch process or as a continuous process, each such step in a one single reactor or in a set of two or more consecutive reactors.

### EXAMPLES

Cleavage of the N-formyl protected amine function of N-(1-hydroxy-2-methyl-propan-2-yl)formamide by catalytic decomposition of the formyl group to carbon dioxide and hydrogen results in the formation of 2-amino-2-methyl-1-propanol

38.3 g of a solution of 15 g N-(1-hydroxy-2-methyl-propan-2-yl)formamide in 35 g water and 50 g tetraethyleneglycol dimethylether (solution having 15% b.w. of formyl protected amine) were continuously pumped through an oil-heated (220° C.) tubular reactor, equipped with 23 mL (16.7 g) of a heterogeneous palladium-based dehydrogenation catalyst (0.75% b.w. palladium on aluminium oxide spheres; BASF SE). A pressure of 20 bara within the reactor was controlled by a RECO-valve. The quantitative analysis of the discharge of the reactor by the means of gas chromatography showed a content of 9.95% 2-amino-2-methyl-1-propanol and 0.6% N-(1-hydroxy-2-methyl-propan-2-yl)formamide. This corresponds to a conversion rate of 96% and a chemical yield of 87%. Given the catalyst volume of 23 mL and an amount of N-(1-hydroxy-2-methyl-propan-2-yl)formamide applied to the reactor of 5.7 g/h, a space-time yield of 0.25 kg/L/h was achieved.

1. A process for converting an N-formyl-protected amine to a corresponding deprotected amine comprising:
deprotecting the N-formyl-protected amine by decomposition of a formyl group to carbon dioxide and hydrogen in a presence of water and a dehydrogenation catalyst and at a temperature in a range from 10 to 300° C.

2. The process according to claim 1, wherein the dehydrogenation catalyst is a heterogeneous catalyst with an active material selected from the group consisting of Pt, Pd, Rh, Ru, Ag, Au, Cu, Ni, Co, Fe, Cr, Mo, W, and V, wherein the active material is in metallic form or as a compound, or a mixture thereof.

3. The process according to claim 2, wherein the heterogeneous dehydrogenation catalyst comprises the active material provided on a support material, selected from the group consisting of activated carbon, aluminium oxide, titanium dioxide, zirconium dioxide, silicon dioxide, niobium oxide, vanadium oxide or a mixture thereof.

4. The process according to claim 1, wherein a weight ratio of the water to the N-formyl-protected amine is in the range from 0.1:1 to 100:1.

5. The process according to claim 1, wherein the deprotection of the N-formyl-protected amine is carried out without addition of hydrogen.

6. The process according to claim 1, wherein the N-formyl protected amine is an N-formyl protected aliphatic primary amine which exhibits one or more hydroxy groups in a 2- (beta), 3- (gamma), or 4- (delta) position related to a carbon atom bearing an N-formyl group (1-position).

7. The process according to claim 1, wherein the N-formyl protected amine is an N-formyl protected aliphatic primary amine which exhibits one or more hydroxy groups in a 2- (beta) position related to a carbon atom bearing an N-formyl group.

8. The process according to claim 1, wherein the N-formyl protected amine is an N-formyl protected amino alcohol compound of a formula I, 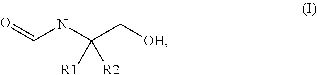
wherein R1 and R2 are independently of each other a hydrogen or an alkyl group having 1 to 4 carbon atoms.

9. A process for manufacturing an amino alcohol compound of a formula II 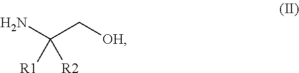
wherein R1 and R2 are independently of each other a hydrogen or an alkyl group having 1 to 4 carbon atoms,
comprising
a) applying hydrogen to a reaction mixture which comprises an N-formyl-protected aminonitrile compound of a formula III 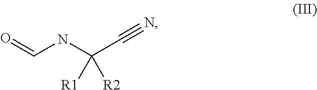
a homogeneous transition metal catalyst and water, to convert the compound of a formula III at least partially to a corresponding N-formyl protected amino alcohol compound of a formula I, 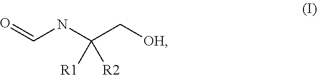
a1) optionally separating the N-formyl protected amino alcohol compound of the formula I resulting from a) at least partially from the rest of the reaction mixture of a), and
b) converting the N-formyl-protected amino alcohol compound of the formula I resulting from a) or a purified N-formyl-protected amino alcohol compound of the formula I resulting from optional a1) to a corresponding deprotected amino alcohol of the formula II according to the process of claim 8.

10. The process according to claim 9, wherein a purification of a1) is mandatory.

11. The process according to claim 10, wherein the purification of a1) comprises a separation of any hydrogen remaining from a).

12. The process according to claim 9, wherein both residues R1 and R2 are methyl groups.
